(19) United States
(12) Patent Application Publication
(10) Pub. No.: US 2012/0266885 A1
(43) Pub. Date: Oct. 25, 2012

(54) NON-INVASIVE VENTILATION MASK AND USE THEREOF

(76) Inventors: Giovanni Guglielmo Landoni, Milano (IT); Luca Cabrini, Agrate Brianza (MI) (IT)

(21) Appl. No.: 13/518,116

(22) PCT Filed: Dec. 23, 2010
(86) PCT No.: PCT/IB10/56058

§ 371 Date: Jun. 21, 2012

Related U.S. Application Data
(60) Provisional application No. 61/290,079, filed on Dec. 24, 2009.

(51) Int. Cl. A61M 16/06

(52) U.S. Cl. 128/205.25

(57) ABSTRACT

The present invention relates to a non-invasive-ventilation-mask to patients with and ongoing endoscopic procedure without interrupting the procedure, without removing the endoscopic probe and without requiring endotracheal intubation.

### FIELD OF THE INVENTION

The present invention relates generally to the field of respiratory devices and methods. More specifically, the present invention discloses a method and apparatus for applying a non-invasive-ventilation-mask to patients with an ongoing endoscopic procedure without interrupting the procedure, without removing the endoscopic probe and without requiring endotracheal intubation.

### BACKGROUND OF THE INVENTION

### Endoscopic Procedures

Technical and medical progress continuously add new properties to endoscopic examinations, like ultra-sonography. Many endoscopic examinations require to pass through the mouth or the nose of the patient to make diagnosis or treatment in a partially non-invasive way.

All hospitals daily perform endoscopic procedures. The most frequently performed procedures are:
TEE (transoesophageal echocardiography) performed by intensive care specialists and cardiologists;
Fiberoptic bronchoscopy with or without intubation performed by pneumonologists, thoracic or ORL surgeons with or without anesthesiologists;
Gastroscopy by gastroscopists (with or without anesthesiologists);
Endoscopic retrograde cholangio-pancreatography (ERCP) performed by gastroscopists (with anesthesiologists)

For example, at the San Raffaele Hospital, Milan, every day at least 15 gastroscopies, 2 ERCP, 5 bronchoscopies and 25 TEE are performed.

As the diagnostic and therapeutic relevance of endoscopic procedures grow, their complexity and their length increase. Therefore more and more high-risk patients, unfitted for surgical interventions or invasive procedures would need endoscopic examinations. The incidence of complications can be high in critically ill patients (1) (2). Patients undergoing endoscopies are frequently in poor conditions, with multiple co-morbidities. Endoscopy-related cardio-respiratory complications have a high incidence in this group of patients.

Therefore, all of these procedures are daily denied to high risk patients (cardiac or pulmonary co-morbidities) or result in complications leading to procedure suspension/delay or in patient intubation.

Furthermore, patient's demand for comfort while undergoing painful or lengthy examinations lead to a widespread use of sedatives. Recently, due to the growing number of endoscopies to be performed under sedation, many endoscopists are tempted to use powerful drugs without the presence of an anesthesiologist. Sedation, in particular, but not only, when administered by a non-anesthesiologist carries the risk of respiratory depression.

Therefore there is the need for a method and apparatus that allow to safely perform endoscopic procedures in high risk patients and in those who require sedation and to prevent intubation and morbidity/mortality in patients who have intraprocedural complications or excessive sedation.

### Non Invasive Ventilation

NIV (non invasive ventilation) is daily performed in all hospitals worldwide. The aim of this technique is to temporary support ventilation and avoid the morbidity and mortality related to tracheal intubation.

Ventilatory support during endoscopies performed through the mouth or the nose is challenging. From an organizational point of view, it is impossible to perform all endoscopies under general anesthesia; furthermore general anesthesia (with tracheal intubation) exposes patients to risks. However, awake procedures are often impossible to perform due to patient discomfort. As a consequence, most endoscopic procedures are performed under sedation.

### Prior Art Masks

Disposable masks with a port dedicated to a probe are commercially available (distributed by VBM Medizintechnik GmbH or patented mask described in U.S. Pat. No. 6,792,943 or US2008/053449).

Such masks must be put on the patient's face before the endoscopy is initiated, to assist in case the patient needs ventilation. Unfortunately, since it is not possible to foresee which patients will really develop respiratory failure, most masks will be wasted.

Furthermore, available masks can be used only for limited kinds of endoscopies, being unfitted for many probes both as port position and diameter. As a matter of fact, no study assessed their use and they are not present in hospitals.

A conventional face mask has a cone-shape canopy with a soft cuff extending around its edge, which is applied against the skin of the patient around the nose and mouth. A port opens into the interior of the canopy so that air or other gases can be supplied to the patient's nose and mouth. Usually, these face masks are held against the face manually or by means of a strap extending around the patient neck/head. Conventional face mask do not usually have a port for endoscopic probes and should be removed to perform endoscopic procedures.

Alternatively, there are masks with a port for endoscopic probes, but these masks should be always placed on the patient's face before starting the endoscopic procedures, without knowing if the patient will require to be ventilated, with increased costs and poor utilization. Conversely, if the patient is under endoscopic examination and subsequently requires ventilation, the examination must be interrupted, the probe(s) must be removed and inserted through the port(s) and then again through the patients' nose or mouth, loosing a considerable amount of time and increasing the risks related to probe insertion since repeating the probe insertion can damage the patient's mucosa or perforate the larynx/pharynx.

A non-invasive ventilatory support by face-mask to be instituted during endoscopic examination is not available, the only possibility being to remove the probe from the patient after interrupting the procedure. Probe removal can be uneasy and the time (and risks) spent to reinsert it in the right position are wasted.

In emergency situations involving patients with cardiopulmonary failure, restless patients or patients with compromised or arrested breathing during endoscopic procedures the probes are removed and the patients ventilated through a mask or a tube to save the patient's life.

The same concepts apply to patients who are denied endoscopic procedures because considered at very high risk of ventilatory failure, or for the costs and organizing problems related to assisted ventilation during the procedures.

The transition from spontaneous breathing with the endoscopic probes to tracheal intubation and repositioning of the endoscopic probes is dangerous and wastes plenty of time: the insertion of the endotracheal tube can take too long and the patient can suffer hypoxia and ventilator associated pneumonia; the repeated insertion of the probes can be dangerous and wastes time.

The typical conventional approach to make this transition involves discontinuing the procedure, completely remove the probe to expose the mouth. The physician inserts a rigid laryngoscope blade into the patient's mouth and then attempts to insert the endotracheal tube through the patient's mouth and into the trachea in the traditional manner. This may require a significant amount of time and the patient may not be breathing sufficiently to maintain adequate blood oxygen levels. In addition, the speed with which the transition process must be completed increases the chances of a mistake being made or unnecessary injury to the patient during the intubation procedure. Even with a cooperative patient, probe insertion and keeping the probe in situ is very uncomfortable and can cause the patient to panic. This procedure can also result in a choking or gagging response that makes the procedure dangerous or impossible.

One common solution to these shortcomings is to sedate the patient during endoscopy. Tranquilizers make the patient more cooperative and less likely to choke, but also tend to suppress the patient's breathing. These side effects are unpredictable and may be unacceptable when dealing with a patient who already suffers from cardiopulmonary complications. Therefore a need exists for an improved device to support ventilation during endoscopy in case of need and that allows the operator to continue the procedure and the patient to be sedated and not to suffer.

None of the prior documents show a mask that incorporates port(s) and that can be applied and used during complicated endoscopic procedures without removing the endoscopic probes.

### DESCRIPTION OF THE INVENTION

The present invention relates to a mask for non invasive ventilation to support ventilation within a few seconds in patients with nasal or oral endoscopic probes without the need of removing them or to interrupt the examination.

Safety, efficacy and patient's comfort are thus improved. There is no risk of respiratory depression, no time wasting, no mask wasting, reduction of hospital costs, the possibility to reach the desired level of sedation without the fear of respiratory complications, the possibility to perform endoscopy in high risk patients that are nowadays denied the procedure and the possibility to reduce the number of general anesthesia performed in these patients.

The mask is suited for all endoscopic probes. The present mask can be used by clinicians (such as but not limited to intensive care specialist, anesthesiologist, gastroenterologists, thoracic surgeons, general surgeons, lung specialist, cardiologists) using an endoscopic probe when acting in intensive units (emergency departments, general or specialized intensive care units), in ordinary wards (hematology, cardiology, thoracic surgery, medicine, etc.) and above all in their own services for in- and outpatients (endoscopic gastroenterology, echocardiography, bronchoscopy and so on). Likely, the mask of the invention will be used in at least the 2-5% of procedures (even at higher percentage for bronchoscopy and ERCP).

The use of the mask of the present invention allows to increase the number of high risk patients on which endoscopic procedures can be performed.

One of the main advantage of the present mask resides in the fact that it is composed by two parts and it can be placed on the patient even if endoscopic probes are already inserted.

It is then an object of the invention a face mask for ventilation of a subject essentially consisting of:
a) two almost symmetrical semi-halves able to assemble along their longitudinal axe, each of them comprising on said longitudinal axe: i) sealing and securing elements able to close the mask when placed on the face of a subject, and ii) symmetrical semi-holes containing suitable gasket means so that one or more holes for endoscopic probes are formed when the mask is closed, and sealing means for said holes if not utilized;
b) one or more holes for ventilator circuit;
c) fastening means to secure the mask to the subject.

Preferably the holes for ventilator circuit are formed when the mask is closed by one or more symmetrical semi-holes along the longitudinal axe.

Alternatively at least one hole for ventilator circuit is present only on one of the two semi-halves.

The holes for endoscopic probes are positioned at a suitable position for mouth and/or nose endoscopic probes.

According to a preferred embodiment, the face mask for ventilation of the invention comprises:
the holes or apertures for endoscopic probes comprising an oral endoscopic or probe port (1) and/or a nasal endoscopic or probe port (2);
the holes for ventilation comprising an upper ventilation hole (3) and a lower ventilation hole (4) for connection with ventilator circuit,
fastening means to patient (5),
fastening means (6) including hooks to fasten lids (15) and gaskets (18) by means of elements (14) or (16), sealing and securing means (7), holding elements (8) for holding sealing and securing means (7), soft material extending around the external edge (9) of the mask, upper and lower assembling means (10), reinforcing elements (11) that may penetrate into suitable cavities in the contralateral hemi-mask (11a).

In a preferred embodiment the gaskets (18) are “C-shaped” rubber gasket.

The two parts of the mask are similar in structure (differ only in the sealing and securing elements or in dimensions) and once sealed together they display one or more holes or aperture. Preferably, one or more holes are for the oxygen/air mixture or for the ventilation, and one or more holes are for the endoscopic probe(s) that are already positioned with the patient. Furthermore, the hole(s) for the oxygen/air mixture or for the ventilation may be placed asymmetrically on one of the two hemisphere of the mask.

Only when ventilation is required the disposable mask is used (in particular, but not exclusively, during emergency situations). As soon as the ventilation is not required any more, the mask can be opened and removed.

The mask is susceptible of improvements, and the aim of the present invention is indeed to provide a face mask that can be placed in emergency conditions on patients with endoscopic probes inserted in the mouth and/or in the nose.

With this aim, the object of the present invention is to provide a face mask that allows to rapidly act on the patient without having to remove the endoscopic probes.

Another object of the present invention is to provide a mask that, while having considerably improved characteristics still has a simplified structure and a competitive cost.

The invention will be now described by means of non limiting examples referring to the following figures:

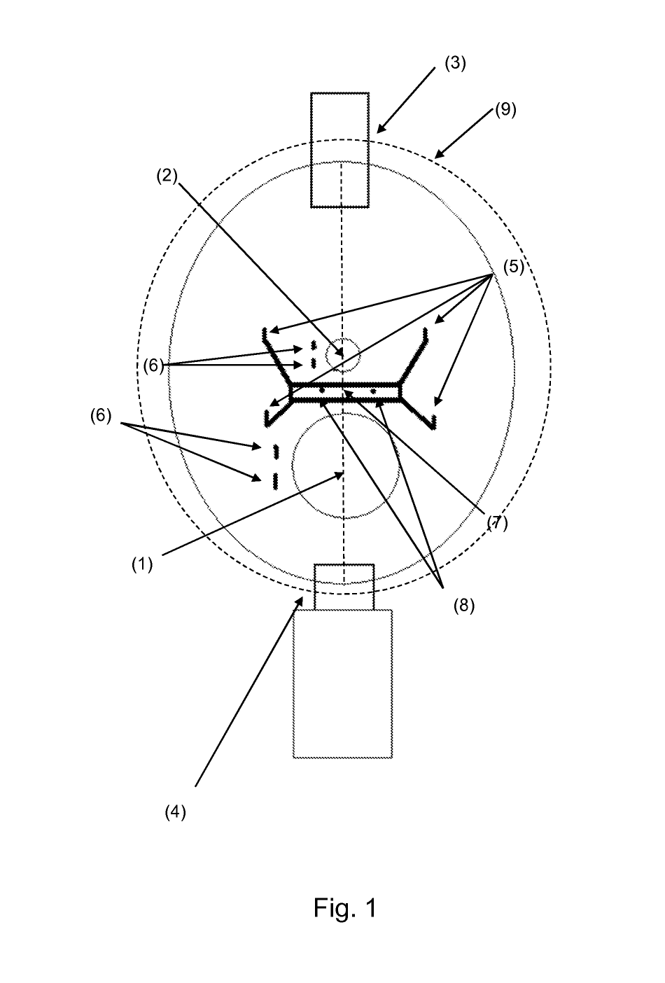

FIG. 1: Front view of the outside portion of the mask when the two hemi-parts are assembled.

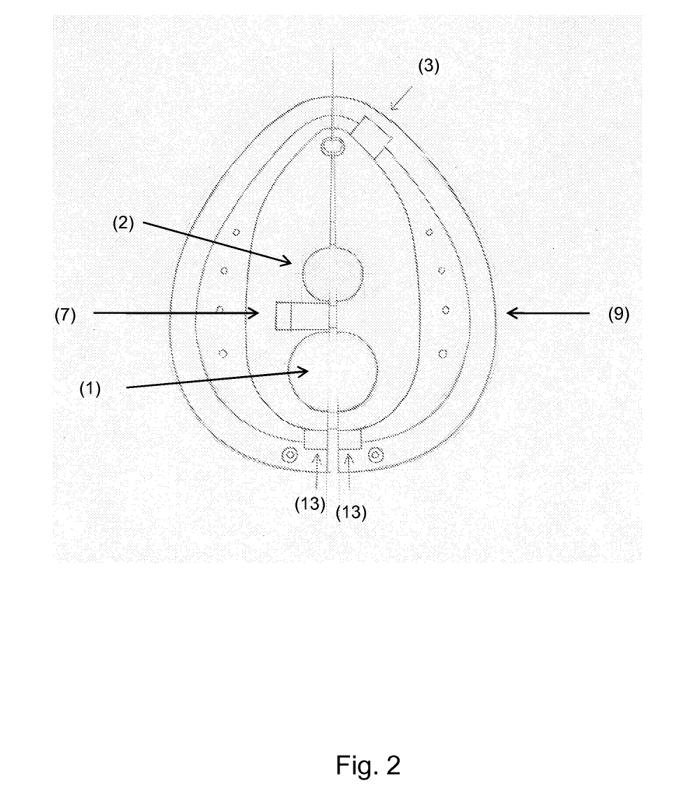

FIG. 2: Simplified front view of the outside portion of the mask when the two hemi-parts are not fully assembled before use.

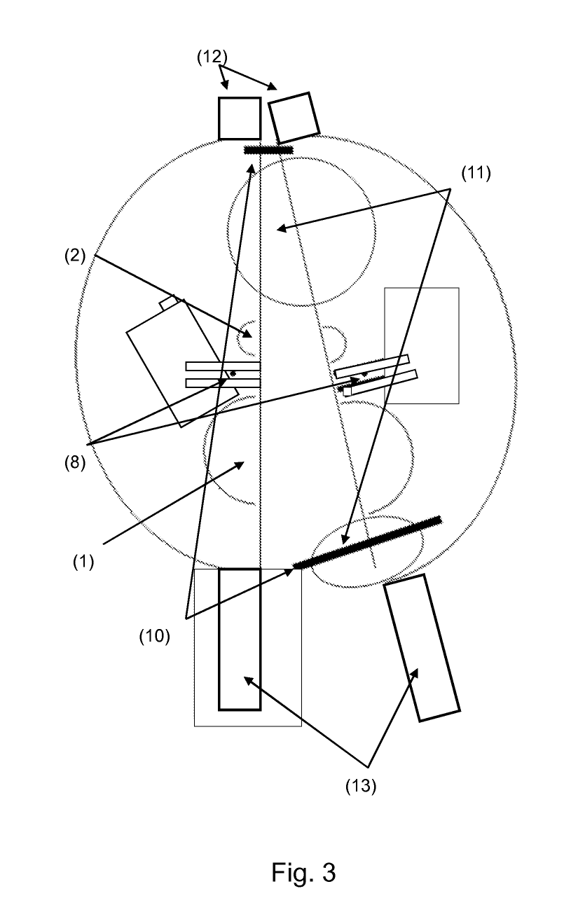

FIG. 3: Detailed front view of the outside portion of the mask when the two hemi-parts are not fully assembled before use.

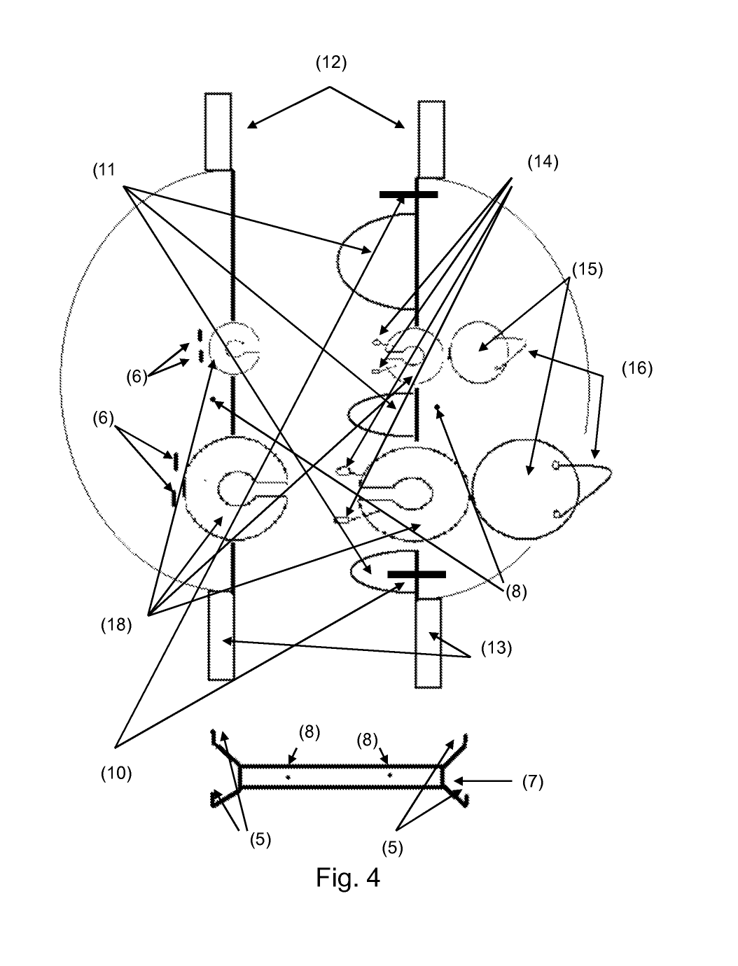

FIG. 4: Detailed front view of the outside portion of the mask when the two hemi-parts are not fully assembled before use. In particular details on connection/disconnection of the hemi-two parts of the mask are shown.

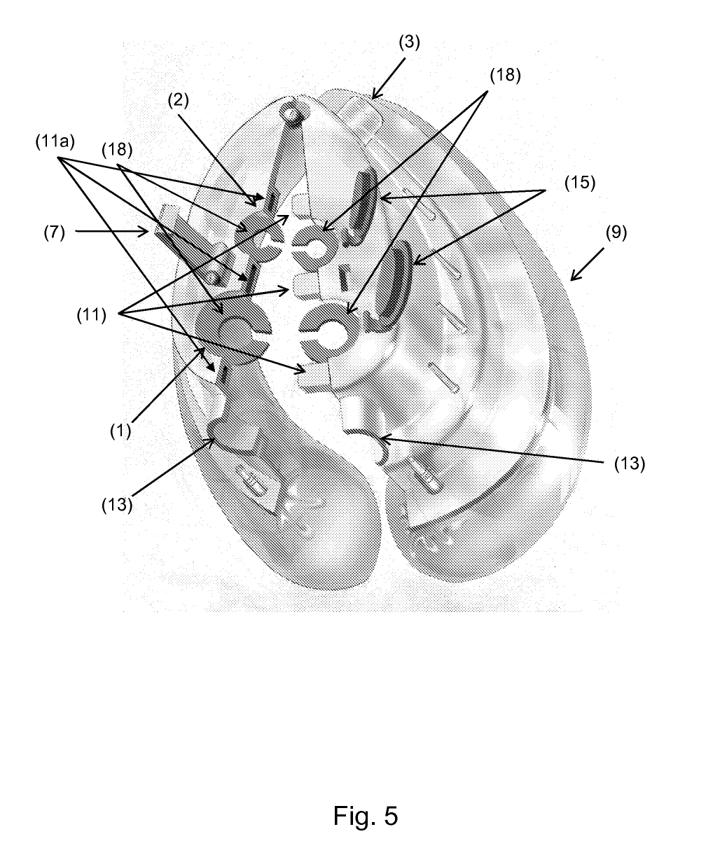

FIG. 5: Perspective outside view of the mask in a semi-open position.

With reference to the figures, a particular embodiment of the mask for ventilation, according to the invention, comprises an oral endoscopic or probe port (1), a nasal endoscopic or probe port (2), an upper ventilation hole (3), a lower ventilation hole (4) for connection with ventilator circuit, fastening means to patient (5), fastening means (6) such as hooks to fasten lids (15) and gaskets (18) by means of elements (14) or (16), sealing and securing means (7) such as plastic bar, holding elements (8) such as pins for holding sealing and securing means (7), soft material extending around the external edge (9) of the mask, upper and lower assembling means (10), reinforcing elements (11) that may penetrate into suitable cavities in the contralateral hemi-mask (11a), symmetrical half-aperture (12) forming the upper ventilation hole (3) when closed, symmetrical half-aperture (13) forming the lower ventilation hole (4) when closed, fastening means (14) such as elastic rings for fastening the gasket (18) to the elements (6), closing means such as plastic lids (15) that close the ports when not in use, fastening means (16) such as elastic bands for fastening (15) to (6), “C-shaped” gasket (18) such as rubber gasket which when superimposed leave a central hole for the endoscopic probes.

The mask has at least one hole or aperture, preferably two or three holes (one for the air/oxygen supply and preferably at least one for the endoscopic probes).

In an embodiment the two separate parts (halves) of the mask are connected by connecting means, allowing the opening and the closure when placed on the patient (FIGS. 2, 3 and 5); alternatively the two separate parts of the mask are completely separated and are assembled only when placed on the patient (FIG. 4). When assembled, the mask is shaped like a conventional face mask. The mask can be fitted to the patient's face before the endoscopic procedure or, most importantly, during the endoscopic procedure and even after.

The two sides of the mask are fixed with mechanical devices and/or with plastic of robbery consistency, forming an effective mechanical bond and gas-tight seal one-each-other. This connection is sufficient to hold the face mask securely for a normal use.

The clinician simply connects the two halves of the mask and the mask to the ventilator to provide ventilation, as required. Alternatively, the patient can be manually ventilated by connecting a resuscitation bag to the proximal end of the mask.

As soon as the ventilation is not required anymore, the mask can be opened and removed.

The face mask does not need necessarily to fit around the openings of both the nose and mouth. It could, for example, fit around only the mouth or around only the nose.

If ventilation is needed again, the mask can be utilized more and more times in the same patient or in different patients if a sterilization process is permitted.

The present invention is susceptible of numerous modifications and variations, all of which are within the scope of the appended claims. All the details may further be replaced with other technically equivalent elements.

The two pieces, hemi-masks, halves or parts are assembled together to obtain a mask (with port for endoscopic probes). The two pieces may be seal-assembled through welding, bonding or any other suitable means. They can have any kind of securing connection. The connection is gas-tight. They can have single or multiple connection points. The connection may occur top, middle or bottom part of the mask, or at all of them.

The two pieces of the mask are connected with a form-fitting connection that holds the mask securely but removably. This connection connects two elements together. When the two contacting surfaces are positioned together, a gas-tight, removable connection occurs. The two parts can be connected to each other in any number of ways. For example, they can be secured with a locking mechanism such as a cotter pin, an elastic band or a C-shaped spring clip. Alternatively, they may be stretched to fit over small protrusion extending sideward from each other surface. In yet another embodiment, the two parts may have threads that screw into corresponding threads in the other part. Other measures for connecting the two parts include, for example, forcing one part into a recess of the other part or applying an adhesive or VELCRO®.

The ports of the mask of the invention are able to accept endoscopes for gastrointestinal and/or pulmonary examination. The ports are positioned on the lower front portion of the mask to allow devices to enter the mouth (or the nose) of a patient, if already wearing the mask. The ports include a flexible, perforated diaphragm to maintain seal integrity required for oxygen therapy purposes.

The mask of the present invention fits over the nose and/or mouth creating a seal and provides a ventilation port that is attached to a ventilator and/or a manual ventilation and a perforated diaphragm to allow passage of an endoscope.

### EXAMPLE 1

Endotracheal intubation under fiberoptic guide is the suggested technique for planned difficult endotracheal intubation in the operating room. Awake intubation is an extremely painful procedure.

PATIENT A didn't receive sedation and died of myocardial infarction during the procedures. Tachycardia and hypertension caused by the procedure initiated myocardial infarction in this patient.

PATIENT B received too much sedation for the procedure and died because of hypoxia and respiratory failure. The sedation caused respiratory arrest in this patient.

PATIENT C was anesthetized and but underwent endotracheal intubation with delay because of difficult airway. He died shortly thereafter (hypoxic brain death). The junior anesthesiologist wasn't confident with the procedure of fiberoptic intubation.

In patient A, the presence of the mask of the invention would have allowed the physician to administer sedation to the patient and prevent myocardial infarction. In patient B, the presence of the mask of the invention would have permitted ventilation in this high risk patient during sedation. In patients C the presence of the mask of the invention would have allowed the junior anesthesiologist to try and safely perform the tracheal intubation with the fiberoptic endoscope.

### EXAMPLE 2

Transoesophageal echocardiography (TEE) is widely used in intensive care units, arrhythmology departments (before electrical cardioversion and/or ablation procedures), cardiac surgery departments (before cardiac surgery), cardiology outpatients.

PATIENT A was denied TEE because suffering of chronic obstructive pulmonary disease and died shortly thereafter of severe aortic stenosis.

PATIENT B received general anesthesia to perform TEE in the suspect of severe aortic stenosis and died because of general anesthesia

PATIENT C was slowly recovering in intensive care unit after major surgery. He received TEE to diagnose pericardial effusion. His clinical condition deteriorated during the procedure, the patient required intubation and died 2 weeks later because of ventilator associated pneumonia.

PATIENT D was planned for percutaneous aortic valve replacement (too high risk for cardiac surgery). The procedure was lengthy and after three hours he became restless and required intubation during TEE. He died of ventilator associated pneumonia in the ICU.

In patient A, the presence of the mask of the invention would have allowed the physician to reach a prompt diagnosis and prevent death. In patient B and C, the presence of the mask of the invention would have avoided ventilator associated pneumonia.

### REFERENCES

1. Shenck J, Muller C H, Lubbers H et al. Does gastroscopy induce myocardial ischemia in patients with coronary heart disease? Endoscopy 2000; 32:373-376
2. Azoulay E, Mokart D, Rabbat A et al. Diagnostic bronchooscopy in hematology and oncology patients with acute respiratory failure: prospective multicenter data. Crit Care Med 2008; 36:100-107.

1. A face mask for ventilation of a subject, comprising:
two almost symmetrical semi-halves able to assemble along longitudinal axes thereof, each of said two almost symmetrical semi-halves comprising sealing and securing elements on said longitudinal axes for closing the mask when placed on the face of a subject and symmetrical semi-holes on said longitudinal axes, said symmetrical semi-holes containing suitable gasket means so that one or more holes for endoscopic probes are formed when the mask is closed, and sealing means for said one or more holes for endoscopic probes if not utilized;
one or more holes for a ventilator circuit;
fastening means to secure the mask to the subject.

2. A face mask for ventilation according to claim 1, wherein said holes for the ventilator circuit are formed when the mask is closed by one or more of said symmetrical semi-holes along the longitudinal axes.

3. A face mask for ventilation according to claim 1, wherein at least one hole for the ventilator circuit is present only on one of the two semi-halves.

4. A face mask for ventilation according to claim 1, wherein the one or more holes for endoscopic probes are positioned at a suitable position for mouth and/or nose endoscopic probes.

5. A face mask for ventilation according to claim 1, wherein:
the one or more holes for endoscopic probes consist of one or more of an oral endoscopic or probe port and a nasal endoscopic or probe port;
the one or more holes for said ventilator circuit consist of an upper ventilation hole and a lower ventilation hole for connection with the ventilator circuit;
the fastening means to secure the mask to the subject comprises a fastening means to the subject, the face mask further comprising:
another fastening means including hooks to fasten lids and gaskets by means of elements, sealing and securing means, holding elements for holding said sealing and securing means, soft material extending around an external edge of the mask, upper and lower assembling means, reinforcing elements that may penetrate into suitable cavities in a contralateral hemi-mask.

6. A face mask for ventilation according to claim 5, wherein the gaskets are “C-shaped” rubber gaskets.
